(19) United States
(12) Patent Application Publication
(10) Pub. No.: US 2019/0350995 A1
(43) Pub. Date: Nov. 21, 2019

(54) THERAPEUTIC COMPOSITION DERIVED FROM ANTHOSTEMA SENEGALENSE

(71) Applicant: LABORATOIRE MICHEL IDERNE, Rosheim (FR)

(72) Inventor: Aliou Mamadou Balde, Bruxelles (BE)

(73) Assignee: LABORATOIRE MICHEL IDERNE, Rosheim (FR)

(21) Appl. No.: 16/476,192

(22) PCT Filed: Oct. 20, 2017
(86) PCT No.: PCT/FR2017/052891

§ 371 Date: Jul. 5, 2019

(30) Foreign Application Priority Data
Oct. 21, 2016 (FR) 16 60230
Jan. 5, 2017 (FR) 17 50080

(51) Int. Cl. A61K 36/47; A61K 31/56; A61P 31/18; A23L 33/11

(52) U.S. Cl. CPC A61K 36/47; A61K 31/56; A61P 31/18; A61K 2236/33; A61K 2236/17; A61K 2236/51; A23L 33/11

(57) ABSTRACT

The present invention relates to a chemical composition characterized in that it comprises:—the molecule A or a physiologically acceptable salt thereof, and/or:—the molecule B or a physiologically acceptable salt thereof, said molecule A having the following formula: and the molecule B having the following formula: the molecule A or a physiologically acceptable salt thereof being biologically active and stimulating immune activity, the molecule B or a physiologically acceptable salt thereof being biologically active and stimulating immune activity.

The present invention falls within the field of chemical compositions that are suitable for use in the preparation of medicinal products or food/dietary supplements.

In particular, the invention makes possible the preparation of medicinal products or food supplements that are capable of enhancing the body's immune system and defences, in particular against viruses, bacteria, fungi, and protozoa that are generally responsible for a number of sometimes life-threatening diseases.

The invention relates specifically to a chemical composition that comprises of biologically active molecules that contribute to increasing and stimulating the immune activity in the body and thus enhance the immune defences, in particular against viruses.

More particularly, the chemical composition of the invention exhibits a biological activity against the “Human Immunodeficiency Virus” (HIV) and its resultant, “Acquired Immune Deficiency Syndrome” (AIDS), generally accompanied by opportunistic infections thereof.

It should be noted that HIV is a retrovirus that infects humans and of which two types are known, HIV-1 and HIV-2. The HIV infection is a pandemic that is widespread across the entire planet, particularly sub-Saharan Africa which alone accounts for 69% of all cases. The HIV infection evolves in a patient in multiple phases,
The first phase, referred to as “primary infection”;
An asymptomatic phase, referred to as “latency,”
An increased immunosuppression phase, referred to as “AIDS”.

The primary infection phase consists of active replication, in the body of HIV which causes destruction of the cells of the immune system in particular T4 lymphocyte cells.

The phase of “latency” represents a phase in which the viral load decreases slightly and is stabilised in the body.

The “AIDS” phase consists of a greater multiplication of HIV which is invasive and wherein the symptoms of AIDS as well as opportunistic diseases appear, eventually culminating in the death of the patient.

To date, there does not exist any effective treatment to cure AIDS and completely eliminate the development and spread of the virus (HIV) in the body of the patients.

In a known matter, various types of treatments such as triple therapy treatments combining three antiviral molecules are used in order to slow the growth and multiplication of HIV in the body and its progression to the AIDS stage.

However, these treatments continue to be very expensive with a high purchase price mainly due to the long lead time for the manufacture and production thereof at industrial scale. Moreover, the latter require very rigorous storage conditions and their access is restricted in respect of patients in developing countries.

Furthermore, current treatments are not always effective in all patients. Indeed, the rapid pace of multiplication and mutation of HIV in the body being such, these treatments lose their effectiveness over time.

In addition, even when they are effective, these treatments have the disadvantage of presenting many undesirable side effects on the health of the patient being treated who consequently will lose quality of life. In fact, current treatments present a certain level of toxicity that is harmful to the patient's body, in particular due to a number of synthetic chemical excipients of non-natural origin that may present the risk of rejection by the body.

Thus, the present invention is aimed at overcoming the drawbacks of the state of the art by providing a chemical composition consisting of an alternative to the above-noted solutions. This alternative must be effective and serve to enable enhancing the body's immune defences, in particular in patients afflicted with HIV, while also minimising the side effects from administering the medication comprising the alternative chemical composition.

In addition, this alternative solution must be accessible to patients in the developing countries, both in terms of production costs and purchase costs, but also with regard to storage of the active product, and ease of administration and use by the patient.

In addition, in order to achieve this objective, the chemical composition needs to be of the lowest toxicity possible to the body and should target only the viruses, bacteria, fungi, and protozoa responsible for pathologies or disease conditions that weakening the immune system. In order to limit the risks of toxicity, it is advantageous for the chemical composition to present a maximum of elements that are natural in origin (naturally sourced), and considered to carry no/low risk of causing harm to health and the environment.

The present invention is aimed at overcoming the drawbacks of the state of the art, by providing a chemical composition comprising:
the molecule A or one of the physiologically acceptable salts thereof,

and/or;
the molecule B or one of the physiologically acceptable salts thereof,
the said molecule A having the following formula:

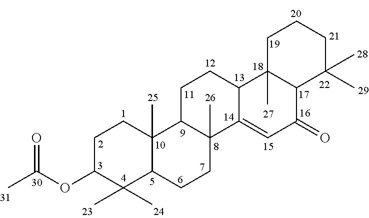

and the molecule B having the following formula:

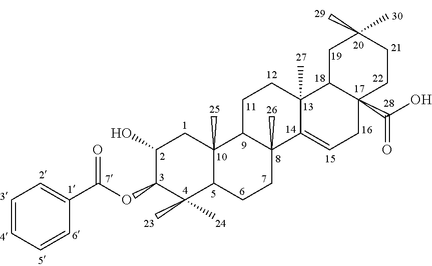

In a specific manner in the chemical composition of the invention:
the molecule A or one of the physiologically acceptable salts thereof is biologically active and is a stimulator of immune activity;
the molecule B or one of the physiologically acceptable salts thereof is biologically active and is a stimulator of immune activity.

Advantageously, the said molecule A presents:
at position 16, as a substitute for the carbonyl function ═O, either an —OH, or an aldehyde carbonyl group —RCOH, or a ketone —R1-CO—R2, or an ester —R—COO—R′, or an amide of the type —R—C(═O)NH2; and/or
at position 3, a glycosylation or esterification in the form of —OR, where “R” is either a feruloyl or caffeoyl group, or a sugar or an amide.

Also advantageously, the said molecule A is conjugated with amino acids, quaternary ammonium salts, glycosides, hemiphtalates, or even carbamates.

Also in an advantageous manner, the said molecule B presents, at position 28, an esterification of an amide or a sugar.

In addition, according to other characteristic features of the invention, the chemical composition of the invention comprises at least one of the following molecules or one of the physiologically acceptable salts thereof, or one of the possible combinations of the said following molecules, the said one or more molecule(s) being selected from the following list:
the molecule C having the formula:

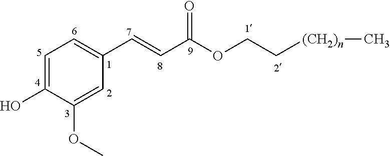

wherein “n” is comprised between 8 and 26:
the molecule D having the formula:

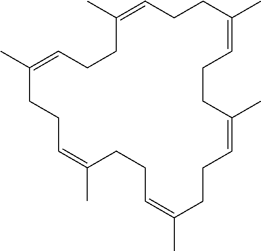

the molecule E having the formula:

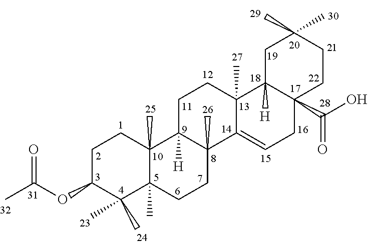

the molecule F having the formula:

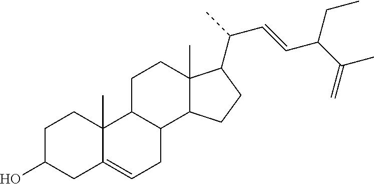 [SMILES: C=C(C)C(/C=C/[C@@H](C)C1CCC2C3CC=C4CC(O)CCC4(C)C3CCC21C)CC]

the molecule G having the formula:

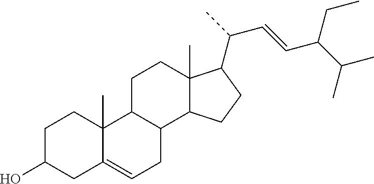 [SMILES: CCC(/C=C/[C@@H](C)C1CCC2C3CC=C4CC(O)CCC4(C)C3CCC21C)C(C)C]

The molecule H having the formula:

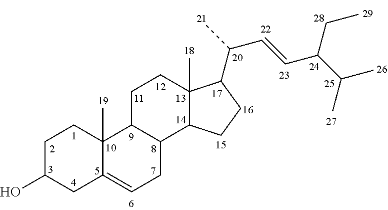 [SMILES: CCC(/C=C/[C@@H](C)C1CCC2C3CC=C4CC(O)CCC4(C)C3CCC21C)C(C)C]

The molecule I having the formula:

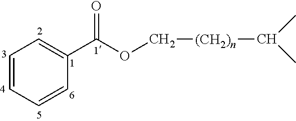

with “n” comprised between 14 and 26;
The molecule J having the formula:

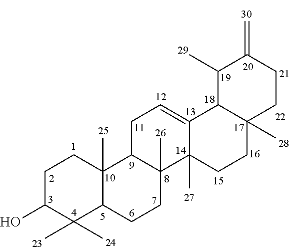

According to a first embodiment of the invention, each of the said molecules A, B, and each of the physiologically acceptable salts thereof is of synthetic origin and derived from a chemical synthesis in the laboratory or from a hemi-synthesis.

According to another characteristic feature of the invention, each of the said molecules A, B and each of the physiologically acceptable salts thereof of the said composition of the invention is of natural origin and derived from a plant extract of Anthostema senegalense.

Advantageously, the plant extract of Anthostema senegalense:
consists of a polar extract, that is aqueous, alcoholic or hydro-alcoholic or an apolar (or non-polar) extract or a mother tincture of Anthostema senegalense with an ethanol content of 70% V/V;
the said plant extract is obtained from the trunk/stem bark, in particular dried bark, of Anthostema senegalense.

Also in an advantageous manner, the said chemical composition of the invention is intended to be used as a medicament or as a food-dietary supplement.

Even more specifically, the said chemical composition of the invention is intended to be used in the treatment of HIV infection type 1 or type 2, AIDS and the clinical manifestations that accompany it.

Preferably, the said chemical composition of the invention is in an oral galenic form or in the galenic form of an infusion sachet.

According to another embodiment, the said composition of the invention is in an oral galenic form of microspheres produced by a method of extrusion and spheronisation, of tablets, soft capsules, granules, gel capsules, powders, ampoules, syrups or even decoctions.

The present invention also relates to an isolation method for isolating by means of extraction, identification and selection of the one or more biologically active molecule(s) of the chemical composition of the invention in which:
A part of the Anthostema senegalense plant is dried;
A plant extract is prepared, from the dried part of the plant, by extraction with an apolar or polar solvent or by maceration, infusion, decoction, percolation, digestion or leaching;
The said plant extract is concentrated under vacuum;
The molecules of the plant extract are separated by chromatography;
In vitro testing is carried out on the biological activity of the said molecules against an HIV-type viral pathogen according to the method of E de Clercq;
The molecules having a selectivity index against the said pathogen ≥50 and a 50% cytotoxic concentration CC₅₀≥45 μg/mL are selected and isolated;
It is then necessary to identify the one or more molecules that have been separated, selected and isolated by means of nuclear magnetic resonance and mass spectrometry.

More specifically for the operational implementation of the said method of the invention:
the part of the Anthostema senegalense plant which is dried consists of the bark of the trunk/stem, or the bark of the roots, or the leaves or the fruits;
a plant extract is prepared by percolation with CHCl3 in order to obtain an apolar plant extract;
after concentration under vacuum, the molecules of the plant extract are separated by means of repeated column chromatography and thin layer chromatography.

In an advantageous manner, in the said method of isolation by extraction of the one or more biologically active molecule(s) that stimulate the immune system, it is necessary to separate the molecules of the said apolar plant extract by column chromatography, using as the stationary phase a polar silica gel and, as the mobile phase, an apolar eluent constituted of a gradient of CHCl₃ in hexane.

Other characteristic features and advantages of the invention shall become apparent from the detailed description that follows of non-limiting embodiments of the invention.

The present invention relates to a chemical composition comprising the molecule A and/or molecule B, or one of the physiologically acceptable salts thereof.

In other words, the said chemical composition comprises the molecule A or one of the physiologically acceptable salts of the molecule A; either in the presence or not of the molecule B or one of the physiologically acceptable salts of the molecule B.

In similar fashion, the said chemical composition comprises the molecule B or one of the physiologically acceptable salts of the molecule B; either in the presence or not of the molecule A or one of the physiologically acceptable salts of the molecule A.

The term “physiologically acceptable” is used to refer to the fact that the one or more molecule(s) as well as their salts do not adversely affect the functioning or the mechanical-, physical-, or biochemical organisation of a living organism, especially of a human.

The chemical composition of the invention is “physiologically acceptable” in its entirety, that is to say that all of the elements that compose it are “physiologically acceptable”. The said chemical composition of the invention is “physiologically acceptable” through its various different components, that is to say, it is biocompatible, entails little or no toxicity for the proper functioning of the body and with no physical or chemical properties that render its administration and metabolism difficult and complicated.

Thus, the composition of the invention is also “pharmaceutically acceptable”, that is to say, it may be used as a medicament to treat a patient and that there are more positive than negative effects on the health of the patient.

The said one or more molecule(s) A and/or B are “biologically active”, in particular against viral, bacterial, and protozoan entities or fungi that are responsible for causing various pathologies. In other words, the molecule A or the molecule B has a single effect or act in a synergistic manner against the proliferation of the said entities that are responsible for causing the said pathologies.

Thus, thanks to the capacity to be “biologically active”, the said one or more molecule(s) A and/or B have the property and effect of stimulating the immune activity in the body and of enhancing it.

According to the invention, the molecule A, a pentacyclic triterpenoid gammacerane, has the following formula:

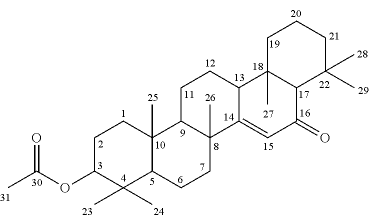

According to one particular embodiment of the invention, the formula of the molecule A may be modified in a manner such that:
At position 16, the carbonyl ═O can be reduced and give an —OH, or even the aldehyde carbonyl functional group —RCOH, a ketone —R¹—CO—R², an ester —R—COO—R′, an amide for example of the type —R—C(═O)NH2;
At position 3, a glycosylation or esterification may be carried out in the form of a functional group of the type 3-OR, wherein “R” may be a feruloyl or caffeoyl group, a sugar or an amide for example of the type —R—C(═O)NH2.

According to another particular embodiment, the molecule A may be conjugated with amino acids, quaternary ammonium salts, glycosides, hemiphtalates or even carbamates.

According to the invention, the molecule B is presented with the following formula:

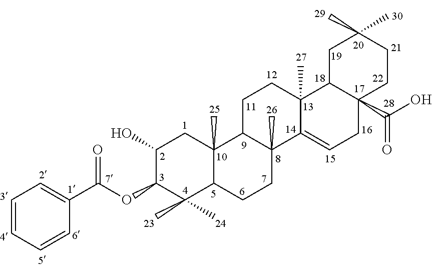

According to one particular embodiment, the molecule B may undergo esterifications at its carbon 28 position in particular with such types of sugars as rhamnose, arabinose, glucose, amino sugars, glucosamine, galactosamine or even with amides of such type as benzylamide, heteroaromatic amide.

For example, for the molecule B, a coupling can be brought about with a side chain of amino acids or peptides among others at its carbon 28.

According to one particular embodiment, the molecule B may undergo modifications at its carbon 3 position, such as:
esterifications with various acids such as ferulic acids or caffeic acids, hydroxycinnamic acids, diméthylsucciniques acids;
conjugations with nucleosides that are inhibitors of reverse transcriptase such as zidovudine, azidothymidine;
bonds with sugar types such as mono or oligosaccharides.

Preferably, the said chemical composition of the invention may comprise, in addition to the molecules A and/or B, one of the following molecules, one of the physiologically acceptable salts thereof, or even one of the possible combinations of the said following molecules:
The molecule C, a ferulic acid ester, having the formula:

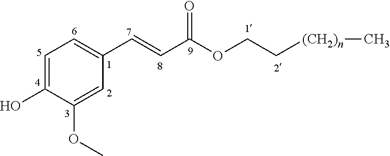

with “n” comprised between 8 and 26;
The molecule D: macrocyclic polyprenoide, having the formula

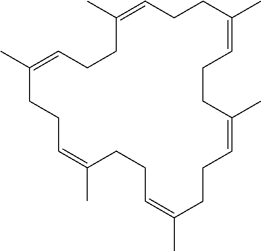

The molecule E: 3-0-acetyl-o-friedo-olean-14-en-28-oic-acid (0-acetyl aleuritolic acid), having the formula:

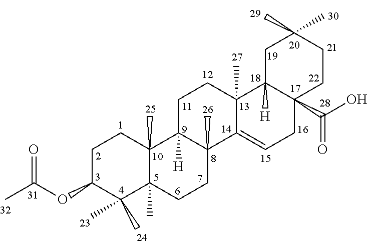

The molecule F: a (3β)-Stigmasta-5,22,25-trien-3-ol having the formula:

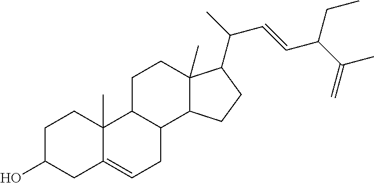 [SMILES: C=C(C)C(/C=C/C(C)C1CCC2C3CC=C4CC(O)CCC4(C)C3CCC12C)CC]

The molecule G, a β-Stigmasterol, having the formula:

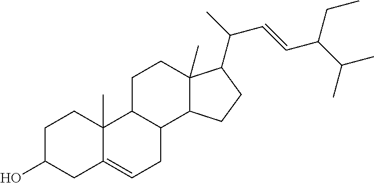 [SMILES: CCC(/C=C/C(C)C1CCC2C3CC=C4CC(O)CCC4(C)C3CCC12C)C(C)C]

The molecule H, a β-Sitosterol, having the formula:

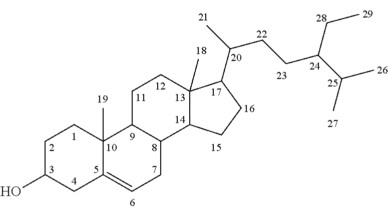 [SMILES: CCC(CCC(C)C1CCC2C3CC=C4CC(O)CCC4(C)C3CCC12C)C(C)C]

The molecule I, an iso-alcohol benzoate, having the formula:

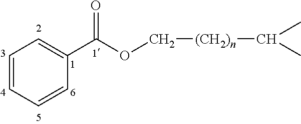

With “n” comprised between 14 and 26;
The molecule J having the formula:

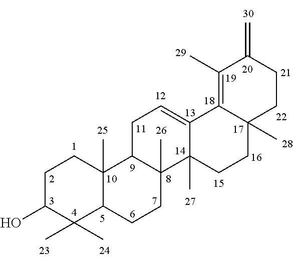

In an advantageous manner and according to one particular embodiment, each of the molecules A to J mentioned above is of natural origin, derived and selected from a plant extract of Anthostema senegalense in particular for its ability and capacity to be biologically active.

Advantageously, the said plant extract is obtained from the bark of the trunk, especially dried, of Anthostema senegalense.

However, other parts of the Anthostema senegalense plant such as the leaves, root bark or fruit can also be used in order to obtain the said plant extract.

Preferably, the said plant extract of Anthostema senegalense consists of a polar extract, that is aqueous, alcoholic or hydro-alcoholic, or an apolar extract, or even a mother tincture of Anthostema senegalense with an ethanol content of about 70% V/V.

Starting from a plant extract of natural origin in order to obtain the biologically active molecules that are stimulators of immune activity has the advantage of rendering them accessible in terms of production costs for the developing countries.

Indeed, Anthostema senegalense is a forest tree widespread across West Africa, in particular in Guinea, Guinea-Bissau, Senegal, Mali, Ivory Coast, Sierra Leone, Benin, Nigeria, etc, which are countries having little means and consequently where the possibility of access to health care is often limited.

In addition, starting from a plant extract of natural origin makes it possible to limit the risks of toxicity and enters an environmentally responsible approach where the molecules of interest are drawn directly from the natural environment.

According to one other embodiment, each of the molecules A to J mentioned above is of synthetic origin, that is to say, derived from a complete chemical synthesis in the laboratory environment or even from a hemi-synthesis.

According to one particular feature of the invention, the said chemical composition of the invention is intended to be used as a medicament or as a food-dietary supplement.

In particular, the said chemical composition of the invention is intended to be used by fatigued individuals whose immune system has been weakened by the presence of a pathogen whose origin is viral, bacterial, fungal, etc.

In an advantageous manner, the applicant was able to demonstrate that the chemical composition of the invention exhibits a biological activity against HIV type 1 and type 2, AIDS and the clinical manifestations that accompany it, as well as certain protozoa in particular Trypanosoma cruzi, Trypanosoma brucei, Plasmodium falciparum.

The biological activity of the chemical composition of the invention is, in particular, due to the presence of the molecules of which it is composed. More specifically, the molecule A, the molecule B, and the molecules C to J are responsible and are at the origin of the biological activity of the chemical composition of the invention. The presence of these molecules A to J in the human body generates the biological activity.

More specifically, the biological activity of the molecules A to J has been demonstrated by means of in vitro tests mentioned later.

Thus, according to the invention, the said chemical composition can be used for the preparation of a pharmaceutical composition that helps to fight against HIV, AIDS, the associated opportunistic pathologies thereof, as well as against the resultant diseases caused by certain protozoa such as trypanosomiasis or malaria.

In addition, the present invention also protects a pharmaceutical composition comprising the chemical composition of the invention, the said pharmaceutical composition being in an oral galenic form.

The term “oral galenic form” is used to refer to the form in which the excipients and active ingredients are to be prepared, the latter being namely the molecules A to J alone or in combination, constituting a medicament for use by oral administration.

According to one particular embodiment of the pharmaceutical composition of the invention, the chemical composition of the invention may be in the form of infusion sachets, this being only when it is completely natural in origin and derived from a plant extract of Anthostema senegalense.

The term “infusion sachet” is used to refer to the form in which the active ingredient is presented, that is to say the molecules A to J derived preferably from a plant extract of Anthostema senegalense. More specifically, an infusion sachet is a small sachet containing a dose of dried plant extract to be infused.

Thus, in the context of the invention, the pharmaceutical composition comprising the chemical composition of the invention may be in the form of a sachet containing a dried plant extract of Anthostema senegalense that one would be able to infuse. In this case, the plant extract of the sachet itself containing at least one of the molecules A or B, is advantageously a combination of molecules A to J.

Preferably, the pharmaceutical composition of the invention is in an appropriate oral galenic form, in particular such as microspheres. These latter are produced by a method of extrusion and spheronisation. The pharmaceutical composition of the invention may also be in a different oral galenic form other than microspheres, in particular in the form of tablets, soft capsules, granules, gel capsules, powders, ampoules, syrups or even decoctions.

The pharmaceutical composition of the invention has the advantage of presenting an effective and less expensive alternative for treating patients afflicted with HIV type 1 or type 2, AIDS or clinical diseases that result therefrom.

Indeed, the oral galenic form or the infusion sachet form of the pharmaceutical composition of the invention facilitates its administration by the patient, requires little or no equipment for administering it and poses no storage problem.

The activity of the one or more molecule(s) of the chemical composition of the invention was demonstrated following the isolation by means of extraction, identification and selection of the said one or more biologically active molecules of interest from an Anthostema senegalense plant.

Accordingly, the present invention also relates to an isolation method for isolating by means of extraction, identification and selection of the one or more biologically active molecule(s) of the chemical composition of the invention.

This method makes it possible to extract from a plant of Anthostema senegalense, the one or more molecule(s) of interest which is/are then considered to be of entirely natural origin. The one or more molecule(s) of interest being the cells exhibiting biological activity against pathogens, in particular a biological activity qualified as “significant”.

In the present application, a “significant” biological activity defines a compound having, against the pathogen, a 50% cytotoxic concentration (CC₅₀)≥45 μg/mL and a selectivity index ≥50.

The said method comprises the following steps:
A part of the Anthostema senegalense plant is dried, for example the bark of the trunk/stem or the leaves;
A plant extract is prepared from the dried part of the plant by means of cold or hot extraction, with a polar or apolar solvent, by means of aqueous extraction, either alcoholic or hydro-alcoholic, or by maceration, infusion, decoction, percolation, digestion or leaching;
The said plant extract is concentrated under vacuum;
The molecules of the plant extract are separated by chromatography;
In vitro testing is carried out on the biological activity of the said molecules against an HIV-type viral pathogen according to the method of E de Clercq;
The molecules having a selectivity index against the said pathogen ≥50 and a 50% cytotoxic concentration CC₅₀≥45 μg/mL are selected and isolated;
It is then necessary to identify the structure of the one or more molecules that have been separated and selected by means of Nuclear Magnetic Resonance and mass spectrometry.

According to one particular embodiment of the method of the invention:
the part of the Anthostema senegalense plant which is dried consists of the bark of the trunk/stem, or the bark of the roots, or the leaves or the fruits;
a plant extract is prepared by percolation with CHCl3 in order to obtain an apolar plant extract;
after concentration under vacuum, the molecules of the said apolar plant extract are separated by means of repeated column chromatography on silica gel, with use as eluent, of a gradient of CHCl₃ in hexane and by means of thin layer chromatography.

The thin layer chromatography may be carried out with use, as mobile phase, of a mixture of dichloromethane/toluene or a mixture of toluene/chloroform or indeed even with chloroform.

Thus, the operational implementation of the method of the invention has made it possible to identify and isolate the molecules having biological activity that are present in a plant extract which is biologically active against HIV. Thereafter, the operational implementation of the method made it possible to select, through the in vitro assay, the molecules that have “significant” biological activity and act as stimulators of the immune system.

The experiments and results detailed here below have provided the applicant with the means to illustrate the immune system stimulating biological activity of the one or more biologically active molecule(s) of the chemical composition of the invention.

1/ In Vitro Testing of the Biological Activity of the Molecules of the Chemical Composition of the Invention

The anti-viral replication efficiency of the molecules of the chemical composition of the invention was tested in vitro against the replication of the viruses HIV type 1 (strain MO and HIV type 2 (strain ROD) in the infected MT-4 cells.

This efficiency was achieved in comparison with three positive control antiretroviral molecules: nevirapine, dideoxycitidine and dideoxyinosine.

More specifically, the antiretroviral activity of the molecules was tested by effectively carrying out the method of E De Clercq in a microplate.

This method consists of producing a cell culture of MT-4 in a microplate, containing as cell culture medium: the medium developed by the Roswell Park Memorial Institute (referred to as “RPMI 1640”), comprising of foetal calf serum, glutamine, sodium bicarbonate, and gentamicin as bacterial inhibitor.

The molecules of the chemical composition of the invention are added at different concentrations into the cell growth medium. Each concentration of molecules to be tested is prepared in triplicate. The MT4 cells placed in the presence of the molecules to be tested are referred to as “treated cells”.

In each cell growth well, a defined amount of HIV-1 or HIV-2 is added in order to obtain the same concentration of virus in each well.

The cells placed in the presence of the HIV are referred to as “infected cells”.

Five days after the start of the process of infecting the MT4 cells with HIV-1 or HIV-2 in the presence of the molecules to be tested at different concentrations, a count is taken of the number of living cells by means of an MTT (tetrazolium salt) colorimetric assay by measuring the optical density at 540 nm.

As negative controls, use is made of cells without HIV 1 or HIV 2, referred to as “non-infected cells”, as well as of cells without the molecules to be tested referred to as “untreated cells”.

Thus, succinctly stated, the method of E De Clercq provides the means to obtain a density measurement at 540 nm for:
the cells infected with HIV-1 or HIV-2, treated with the molecules of the composition of the invention;
the cells not infected with HIV, treated with the molecules of the composition of the invention;
the cells infected with HIV-1 or HIV-2, not treated with the molecules of the composition of the invention;
the cells not infected with HIV, not treated with the molecules of the composition of the invention.

In particular, the effectiveness of the molecules of the composition of the invention was tested:
by measurement and calculation of the (median) 50% inhibitory concentration (IC₅₀);
by measurement and calculation of the 50% cytotoxic concentration (CC₅₀);
by measurement and calculation of the 50% effective concentration dose (EC₅₀); and
by measurement and calculation of the selectivity index (SI), of the fractions containing the said molecules in the presence of the two strains of HIV.

The IC₅₀ is the concentration of the molecule that makes it possible to decrease by 50% the viral proliferation of the negative control in vitro for a given time period, that is to say of the HIV without substance, only in its growth culture medium.

The CC₅₀ is the concentration of active molecules that makes it possible to decrease by 50% the cytopathic effect of HIV on the infected cells. In other words, it is the concentration of active molecules that makes it possible to decrease by 50% the viability of cells.

The EC₅₀ corresponds to the concentration of molecules necessary in order to ensure that 50% of the cells growing in the medium do not submit to the action of the virus, that is to say, that they are protected from the cytopathic effect of the virus.

The EC50 is calculated using the following formula:

[(OD_{T})HIV⁺]−[(OD_{C})HIV⁺]/[(OD_{T})HIV⁻]−[(OD_{C})HIV⁻]×100

Where:
(OD_{T})HIV⁺ represents the optical density measurement at 540 nm, 5 days after infection, for the cells infected with HIV and treated with the composition of the invention, that is to say in the presence of molecules of the composition of the invention;
(OD_{C}) HIV⁺ represents the optical density measurement at 540 nm, 5 days after infection, for the cells infected with HIV in the absence of the molecules of the composition of the invention;
(OD_{T}) HIV⁻ represents the optical density measurement at 540 nm, 5 days after infection, for the cells not infected with HIV and treated with the composition of the invention, that is to say in the presence of the molecules of the composition of the invention; i(OD_{C}) HIV⁻ represents the optical density measurement at 540 nm, 5 days after infection, for the cells not infected with HIV in the absence of the molecules of the composition of the invention.

The selectivity index represents the ratio CC₅₀/EC₅₀. In the context of this application, it will be considered that the biological activity against the pathogen, preferably HIV-1 and HIV-2, is significant and that there is a biological activity that stimulates immune activity, when the selectivity index is 50 and the CC₅₀≥45 μg/ml.

The results are illustrated in Table 1 here below which shows the average values obtained over the duplicates for IC50, CC50 and SI:

| | HIV-1 | | | HIV-2 | | |
|---|---|---|---|---|---|---|
| | IC 50 | CC 50 | | IC 50 | CC 50 | |
| | (ug/mL) | (ug/mL) | SI | (μg/ml) | (μg/ml) | SI |
| sample tested | | | | | | |
| AsC4.2 | 4.130 | 47.83 | 12 | 0.467 | 47.83 | 102 |
| Asc4.5 | 0.866 | 58.48 | 68 | 0.065 | 58.48 | 904 |
| AsC4.6 | 2.350 | 53.33 | 23 | 0.142 | 53.33 | 376 |
| AsC4.7 | 7.670 | 54.08 | 7 | 0.539 | 54.08 | 100 |
| Nevirapine | 0.050 | 0.011 | >4 | >4 | >4 | <1 |
| Dideoxycitidine | 0.16 | 0.12 | >20 | 0.19 | 0.11 | >20 |
| Dideoxyinosine | 2.09 | 0.68 | >50 | 3.78 | 1.22 | >50 |

It is found that the molecules present in the fractions AsC4.2, AsC4.5, AsC4.6, and AsC4.7 possess a significant biological activity against HIV-1 and HIV-2.

In particular, the molecules present in the fraction Asc4.5 exhibit a significant activity against HIV type 1 and type 2.

The molecules present in each of the fractions AsC4.2, AsC4.5, AsC4.6 and AsC4.7 have significant biological activity against HIV type 2. The IC₅₀ of the molecules contained in each of these fractions is better than that of the positive controls of Nevirapine and Dideoxyinosine. Thus, the molecules of these fractions act synergistically to fight against viral multiplication.

In particular, the molecules of the fractions AsC4.5 Asc4.6 and have a high biological activity, that is better than those of the antiretrovirals Nevirapine, Dideoxycitidin and dideoxyinosine, against HIV type 2.

The isolation, identification and selection of the molecules with significant biological activity of the chemical composition of the invention, derived from the said “biologically active” fractions are described here below.

2/ Preparation of the Fractions, Isolation and Selection of the Biological Activity Exhibiting Molecules of the Invention:

From a crude extract of bark of the trunk of an Anthostema senegalense plant, a polar plant extract is prepared by means of percolation with liquid CHCl₃. The said polar plant extract is concentrated under vacuum.

Then column chromatography is carried out with, use as eluent, of a polarity gradient of CHCl₃ in hexane, in order to separate the component constituents according to their charge. At the conclusion of the chromatography, ten fractions are obtained which are respectively named AsC1 to AsC10. A thin layer chromatography process is carried out with each of the ten fractions ASC1, ASC2, ASC3, AsC4, AsC5, AsC6, AsC7, AsC8, AsC9, Asc10 mentioned above, in order to detect the number of potentially different molecules of each of the fractions.

In order to demonstrate in respect of each of the fractions, their biological activity, in particular acting to stimulate the immune system, each of the fractions was tested in vitro vis-à-vis HIV type 1 and HIV type 2, according to the method explained later.

The fraction AsC4 showed the best anti-HIV activity. This fraction AsC4 therefore contains an assembly of molecules with significant biological activity against HIV.

This fraction AsC4 has, as a consequence, been separated by means of column chromatography until 8 fractions were obtained, labelled respectively from AsC4.1 to AsC4.8.

The anti-HIV activity was tested in vitro according to the method of Clerq E on each of the abovementioned fractions AsC4.1, AsC4.2, AsC4.3, AsC4.4, AsC4.5, AsC4.6, AsC4.7 and AsC4.8.

The fractions AsC4.5; AsC4.6; AsC4.2 and AsC4.7 showed the best antiviral activities as illustrated in Table 1 mentioned above.

Thus, the molecules present in majority in these fractions are responsible for the anti-HIV biological activity.

More specifically, the fraction AsC4.5 exhibits a “significant” biological activity pursuant to the meaning of the invention against HIV-1 and greater “significant” biological activity against HIV-2.

Also more specifically, against HIV-2, all of the fractions AsC4.5; AsC4.6; AsC4.2 and AsC4.7 exhibit a “significant” biological activity pursuant to the meaning of the invention.

A sample of each of these fractions AsC4.5 and AsC4.6 was separated by silica gel (SiO2) chromatography, then the molecules were identified by NMR (Nuclear Magnetic Resonance) and Mass Spectrometry.

The results show a predominant ie majority presence in each of these samples AsC4.5 and AsC4.6, of the said molecule A and of the said molecule B, in addition to a series of other molecules with a minority presence.

Thus, the molecule A and the molecule B are envisaged as being responsible for the “significant” biological activity for each of the samples AsC4.5 and Asc4.6.

In order to confirm this opinion, other analyses were performed.

In particular, all of the fractions AsC4.5, AsC4.6, AsC4.2 and AsC4.7 exhibiting anti-HIV biological activity were mixed and separated on silica gel SiO₂ column chromatography on a repeated basis (at least six times), with use as eluent, of toluene/CHCl₃ creating a polarity gradient in order to obtain the sub-fractions referred to as AsC4.5.1 to AsC4.5.7.

Each of the sub-fractions AsC4.5.1 to AsC4.5.7 was then subjected to an in vitro assay as described here above on the viruses HIV 1 and HIV 2. The results of the tests showed a significant inhibitory activity against these two strains for each of the sub-fractions tested.

Each of the abovementioned sub-fractions Asc4.5.1, AsC4.5.2, AsC4.5.3, AsC4.5.4, AsC4.5.5, AsC4.5.6, and AsC4.5.7 was separated and purified on preparative thin layer chromatography by making use of different eluents as the mobile phase.

The eluents used as mobile phase are as follows:
a mobile phase of CH₂Cl₂/toluene in a volume to volume ratio of 1:2;
a mobile phase of toluene/CHCl₃ in a volume to volume ratio of 4:1;
a mobile phase of toluene/CHCl₃ in a volume to volume ratio of 1:1;
a mobile phase of 100% CHCl₃.

More than ten different compounds were obtained for all of the different mobile phases used. Each compound was individually tested against HIV-1 and HIV-2 according to the in vitro method mentioned above.

In a specific manner, among all the compounds, only the compounds cited here below have shown a “significant” biological activity pursuant to the meaning of the invention against the proliferation of HIV-1 or HIV-2.
The compounds AsC4.5.1.1 and R.3.6.6.1, obtained with a mobile phase of CH₂Cl₂/toluene in a volume to volume ratio of 1:2;
The compound AsC4.5.2.1, obtained with a mobile phase of toluene/CHCl₃ in a volume to volume ratio of 4:1;
The compound AsC4.5.4.1, obtained with a mobile phase of toluene/CHCl₃ in a volume to volume ratio of 1:1;
The compounds AsC4.5.5.1, AsC4.5.6.1, AsC4.5.7.1 and AsC4.5.7.2, obtained with a mobile phase of 100% CHCl₃.

In an advantageous manner, the compounds AsC4.5.1.1 and AsC4.5.7.2 exhibit an inhibitory activity that is at least two times greater against HIV-1 and HIV-2 than the other components identified as having a “significant” biological activity.

The identification of all of the compounds with “significant” biological activity as mentioned here above was performed by means of nuclear magnetic resonance and mass spectrometry.

After identification, it turns out that the compounds identified which possess a biological activity against HIV-1 and HIV-2 are constituted of the molecules A to J cited above.

In a more precise manner, it was noted that the compounds AsC4.5.1.1 and AsC4.5.7.2 correspond respectively to the molecule A and the molecule B claimed in the invention. These two molecules A and B are, as a consequence, indeed responsible for significant inhibitory effect on the proliferation of HIV-1 and HIV-2.

In addition, in the fractions AsC4.5 and AsC4.6 that are most active against HIV-1, it was noted that the two molecules A and B are predominantly represented and in high concentrations.

In similar fashion, and still against HIV-1, in the active fraction AsC4.2 the molecule A is predominantly found (majority presence) but not the molecule B, and in the active fraction AsC4.7 the molecule B is predominantly found (majority presence) but not the molecule A.

As a consequence, the technical effect of inhibiting proliferation of HIV 1 or HIV 2 in respect of the fractions having “significant” inhibitory activity is due to the specific presence and action of the molecules A and/or B.

The molecule A as well as the molecule B possess an activity serving to inhibit the proliferation of HIV-1 or HIV-2, each of the molecules A or B being capable of stimulating the immune activity of an infected organism or body.

In the light of these results, the molecules A and B alone or in combination have been specially selected and chosen by the applicant, from among the molecules A to J for which an inhibitory activity against HIV-1 and HIV-2 has also been observed, in order to include them in the chemical composition of the invention that biologically stimulates immune activity.

It follows from these studies that each of the molecules A to J of the invention isolated by means of the above noted techniques exhibit a synergistic inhibitory activity against the proliferation of HIV type 1 and HIV type 2 within cells infected therewith. The biological activity of each of these molecules taken individually or in combination against HIV-1 or HIV-2 has been demonstrated through these experiments.

More specifically, the molecules A and B being represented predominantly (majority presence) in the fractions having inhibitory activity against the proliferation of HIV-1 and HIV-2, are considered to be responsible for the principal and majority biological activity against viruses, in particular HIV 1 or HIV 2.

Further studies of the biological activity of the molecules A and B, taken alone or in combination have shown that the biological activity against HIV is mainly due to the presence of these two molecules. In particular, the presence of the molecule A and/or B in a chemical composition thus has the effect of inhibiting the viral proliferation of HIV-1 and HIV-2 and therefore of biologically stimulating the immune system.

That is why, in implementing an inventive approach, the applicant has specifically selected the molecules A and/or B from the group of molecules A to J having a biological activity, since these two molecules A and B exhibit the significant effect expected against the proliferation of HIV-1 and HIV-2 and provide the ability to stimulate immune activity.

The results of experiments have shown that both the molecule A alone, the molecule B alone, or the combination of the two molecules A and B, has/have a significant biological activity against HIV-1 or HIV-2.

In addition, the significant biological activity is enhanced in the presence of the two molecules A and B. As a result, there is indeed synergy between the two molecules in respect of the activity and the effect thereof against HIV-1 or HIV-2. In other words, a synergy exists between these two molecules so as to enhance their respective inhibitory effect against the proliferation of HIV-1 and HIV-2.

In an advantageous manner, the chemical composition of the invention provides an effective alternative, one that is less expensive, and accessible to developing countries, is easy to produce where it is derived from natural plant origin, which is biologically active, and stimulates the immune activity.

In particular, the chemical composition of the invention may be used in pharmaceuticals domain in the treatment of infection by the HIV virus, as well as AIDS and the clinical manifestations that accompany it.

In addition, the chemical composition of the invention may also be used as a food/dietary supplement that provides the means to stimulate immune activity and the natural immune defences of humans who may be immunocompromised.

1. A chemical composition comprising at least one selected from the group consisting of:
a molecule A or one of physiologically acceptable salts thereof;
and
a molecule B or one of physiologically acceptable salts thereof,
the molecule A having the following formula: 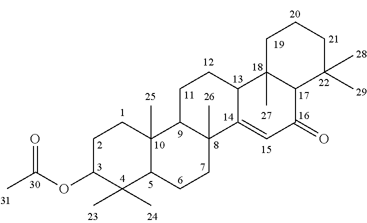
and said molecule B having the following formula: 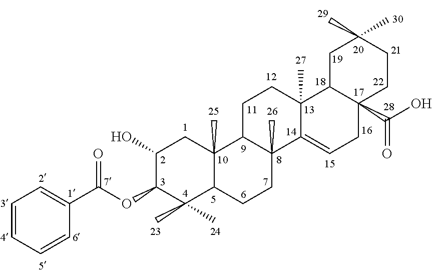
wherein the molecule A or one of the physiologically acceptable salts thereof is biologically active and is a stimulator of immune activity;
wherein the molecule B or one of the physiologically acceptable salts thereof is biologically active and is a stimulator of immune activity.

2. The chemical composition according to claim 1, wherein the molecule A has at least one selected from the group consisting of:
at position 16, as a substitute for the carbonyl function ═O, either an —OH, or an aldehyde carbonyl group —RCOH, or a ketone —R1-CO—R2, or an ester —R—COO—R′, or an amide of the type —R—C(═O)NH2; and
at position 3, a glycosylation or esterification in the form of —OR, where “R” is either a feruloyl or caffeoyl group, or a sugar or an amide.

3. The chemical composition according to claim 1, wherein the molecule A is conjugated with at least one selected from the group consisting of amino acids, quaternary ammonium salts, glycosides, hemiphtalates and carbamates.

4. The chemical composition according to claim 1, wherein the molecule B has, at position 28, an esterification of an amide or a sugar.

5. The chemical composition according to claim 1, wherein the composition further comprises at least one of the following molecules or one of physiologically acceptable salts thereof, or one of possible combinations of the following molecules, the one or more molecule(s) being selected from the following list:
molecule C having the formula: 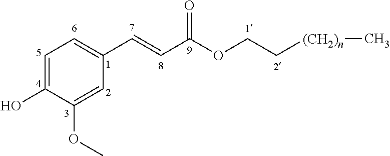
wherein “n” is comprised between 8 and 26;
molecule D having the formula: 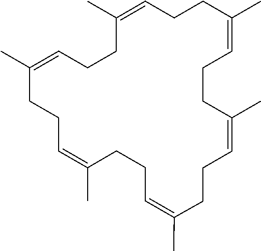
molecule E having the formula: 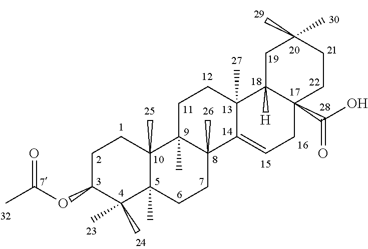
molecule F having the formula: 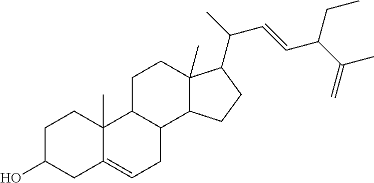 [SMILES: C=C(C)C(/C=C/C(C)C1CCC2C3CC=C4CC(O)CCC4(C)C3CCC12C)CC]
molecule G having the formula: 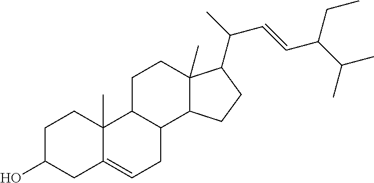 [SMILES: CCC(/C=C/C(C)C1CCC2C3CC=C4CC(O)CCC4(C)C3CCC12C)C(C)C]
molecule H having the formula: 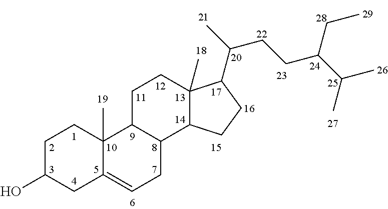 [SMILES: CCC(CCC(C)C1CCC2C3CC=C4CC(O)CCC4(C)C3CCC12C)C(C)C]
molecule I having the formula: 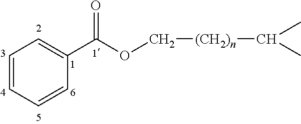
wherein “n” is comprised between 14 and 26;
molecule J having the formula:

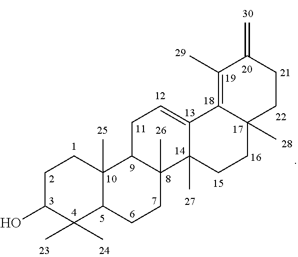

6. The chemical composition according to claim 1, wherein each of the molecules A, B, and each of the physiologically acceptable salts thereof is of synthetic origin and derived from a chemical synthesis in the laboratory or from a hemi-synthesis.

7. The chemical composition according to claim 1, wherein each of the molecules A, B and each of the physiologically acceptable salts thereof is of natural origin and derived from a plant extract of Anthostema senegalense.

8. The chemical composition according to claim 7, wherein the plant extract of Anthostema senegalense consists of a polar extract, that is aqueous, alcoholic or hydro-alcoholic or an apolar or non-polar extract or a mother tincture of Anthostema senegalense with an ethanol content of 70% V/V.

9. The chemical composition according to claim 8, wherein the plant extract is obtained from the trunk/stem bark, in particular dried bark, of Anthostema senegalense.

10. The chemical composition according to claim 1, which is in the form of a medicament or a food-dietary supplement.

11. A pharmaceutical composition comprising the chemical composition according to claim 1, which is formulated for the treatment of at least one selected from the group consisting of HIV infection type 1, HIV infection type type 2, AIDS, and clinical manifestations that accompany AIDS.

12. The pharmaceutical composition according to claim 11, which is in an oral galenic form.

13. The pharmaceutical composition according to claim 11, which is in an oral galenic form of microspheres produced by a method of extrusion and spheronisation, tablets, soft capsules, granules, gel capsules, powders, ampoules, syrups or decoctions.

14. An isolation method for isolating by extraction, identification and selection of the one or more biologically active molecule(s) of the chemical composition according to claim 1, the method comprising:
drying a part of the Anthostema senegalense plant to obtain a dried part of the plant;
preparing a plant extract from the dried part of the plant, by extraction with an apolar or polar solvent or by maceration, infusion, decoction, percolation, digestion or leaching;
concentrating the plant extract is concentrated under vacuum;
separating molecules of the plant extract by chromatography;
carrying out in vitro testing is on the biological activity of the molecules against an HIV-type viral pathogen according to the method of E de Clercq;
selecting and isolating molecules having a selectivity index against the pathogen ≥50 and a 50% cytotoxic concentration CC₅₀≥45 μg/mL;
identifying the one or more molecule(s) that have been separated, selected and isolated by means of nuclear magnetic resonance and mass spectrometry.

15. The method according to claim 14, wherein:
the part of the Anthostema senegalense plant which is dried consists of the bark of the trunk/stem, or the bark of the roots, or the leaves or the fruits;
the plant extract is prepared by percolation with CHCl3 in order to obtain an apolar plant extract;
after concentration under vacuum, the molecules of the plant extract are separated by repeated column chromatography and thin layer chromatography.

16. The method according to claim 15, wherein the molecules of the apolar plant extract are separated by repeated column chromatography, using as stationary phase a polar silica gel and, as mobile phase, an apolar eluent constituted of a gradient of CHCl₃ in hexane.

17. The chemical composition according to claim 2, wherein the molecule A is conjugated with at least one selected from the group consisting of amino acids, quaternary ammonium salts, glycosides, hemiphtalates and carbamates.

18. The chemical composition according to claim 17, wherein the molecule B has, at position 28, an esterification of an amide or a sugar.

19. The chemical composition according to claim 2, wherein the molecule B has, at position 28, an esterification of an amide or a sugar.

20. The chemical composition according to claim 3, wherein the molecule B has, at position 28, an esterification of an amide or a sugar.
